(19) United States
(12) Patent Application Publication
(10) Pub. No.: US 2023/0125408 A1
(43) Pub. Date: Apr. 27, 2023

(54) SUBSTANCE DELIVERY MASK

(71) Applicant: NUECOLOGY BIOMEDICAL INC., RICHMOND (CA)

(72) Inventors: CHUNG CHIN SUN, RICHMOND (CA); DEAN MO LIU, SURREY (CA)

(21) Appl. No.: 17/937,844

(22) Filed: Oct. 4, 2022

Related U.S. Application Data
(60) Provisional application No. 63/270,216, filed on Oct. 21, 2021.

(51) Int. Cl. A61M 16/16; A61M 11/04; A61M 16/00

(52) U.S. Cl. CPC A61M 16/164; A61M 11/04; A61M 16/022; A61M 2205/8206; B32B 5/022

(57) ABSTRACT

A substance delivery mask includes a body, an exhaling valve, an inhaled valve, and a fog module. The fog module comprises a controlling module, a container, and an atomizer. The controlling module is disposed on the body. The container is disposed on the controlling module, the container is storing a mixed liquid, and the mixed liquid includes water and a plurality of nanoparticles. The atomizer is disposed on the container, the atomizer is used to atomize the water of the mixed liquid to form fog particles, and the fog particles are wrapped several of the nanoparticles respectively. The substance delivery mask can encapsulate the water-soluble or water-insoluble medicines, or nutritional products in the fog particles generated from the atomizer. The present invention can deliver medicine, nutritional products or vaccines by inhalation or oral.

This application claims priority of U.S. Provisional Application No. 63/270,216 filed on 21 Oct. 2021 under 35 U.S.C. § 119(e); the entire contents of all of which are hereby incorporated by reference.

### BACKGROUND OF THE INVENTION

### Field of the Invention

The present invention relates to a substance delivery mask, particularly a substance delivery mask that can deliver medicine to the user taken by inhalation or oral.

### Description of the Related Art

The treatment of toxic viruses has been a major challenge in clinical practices. The global pandemic crisis of novel coronavirus disease (COVID-19) caused by severe acute respiratory syndrome coronavirus 2 (SARS-CoV-2) began in December 2019 has since spread worldwide. As of the end of August 2021, there have been more than 200 million reported cases and more than 3 million deaths in more than 200 countries, and it appears to be continuously worsened. This novel Beta coronavirus is similar to severe acute respiratory syndrome coronavirus (SARS-CoV) and Middle East respiratory syndrome coronavirus (MERS-CoV). Based on its genetic proximity, it likely originated from bat-derived coronaviruses with spread via an unknown intermediate mammal host to humans. The viral genome of SARS-CoV-2 was rapidly sequenced to enable diagnostic testing, epidemiologic tracking, and the development of preventive and therapeutic strategies.

Currently, there is no strong and clinically-decisive evidence from randomized clinical trials that any potential therapy improves outcomes of inpatients with either suspected or confirmed COVID-19. Further, it may be necessary to filter the ambient air supplied to the patient because the ambient air is likely to contain a virus or bacteria. It may also be necessary to filter the exhaled breath of the patient (or non-patient who might be asymptomatic virus carriers) before the exhaled breath is released into the atmosphere. For example, the patient has a medical condition that may result in a virus or bacteria being exhaled and likely to harm a person nearby. Except for the use of medication, such as antiviral drugs or vaccines, masks have been widely used to effectively and potentially block inhaled viruses, which may be in the form of aerosol microparticles, larger particles, or a free-standing virus itself. The virus from exhaled breath of an infectious person has a size of approximately 100 nm.

Besides those antiviral drugs or vaccines available, their protection is limited to the areas inside the body system, rather than those respiratory tracts including nasal, throat, and lung even goes to the GI tract, where those epithelial cells or mucous membranes along with those organs and tissues may become the virus colonies and put a subsequent life threatening risk to the asymptomatic virus carrier or other nearby people.

However, conventional masks cannot deliver encapsulated water soluble/insoluble medicines. Therefore, there is a need to improve the reliability of conventional masks.

### SUMMARY OF THE INVENTION

In view of the shortcomings of the prior art, the object of the present invention is to provide a substance delivery mask utilizing the atomizer to form the fog particles carrying medicines and nutritional products. The substance delivery mask can deliver medicine by inhalation or oral.

The substance delivery mask of the present invention comprises a body, an exhaling valve, an inhaled valve, and a fog module. The body has a first surface and a second surface opposite to each other. The exhaling valve is disposed on the body, a first end of the exhaling valve is exposed to the first surface, and a second end of the exhaling valve is exposed to the second surface. The inhaled valve is disposed on the body, a first end of the inhaled valve is exposed to the first surface, and a second end of the inhaled valve is exposed to the second surface. The fog module comprises a controlling module, a container, and an atomizer. The controlling module is disposed on the first surface. The container is disposed on the controlling module, and the container is storing a mixed liquid. The mixed liquid comprises water and a plurality of nanoparticles. The atomizer is disposed on the container. The atomizer is atomized in the water of the mixed liquid to form a plurality of fog particles, and the fog particles are wrapped in several of the nanoparticles respectively.

In an embodiment of the present invention, the body is made of airproof material, such as silicon, rubber, plastic, or a combination thereof.

In an embodiment of the present invention, the body is made of a breathable material, such as polypropylene nonwoven material, melt-blown nonwoven material, and composite nonwoven material.

In an embodiment of the present invention, the controlling module comprises a battery, a programming module, and a switch. The battery is connected with the programming module, and the switch connects between the battery and the programming module.

In an embodiment of the present invention, the first end of the inhaled valve is connected with the controlling module, and the second end of the inhaled valve is connected with the body.

In an embodiment of the present invention, each of the nanoparticles is an AGO particle.

In an embodiment of the present invention, each of the AGO particle is coated the medicines, such as a remdesivir, a dexamethasone, a favipiravir, an avigan, a hydroxychloroquine, a carfilzomib, a darunavi, a pitavastatin, a lamivudine, a lopinavir, a nelfinavir, a ritonavir, a darunavir, a ledipasvir, a telaprevir, a rosuvastatin calcium, an atovaquone, a moexipril, an azithromycin, a curcumin, an artemisinin, a xiaoqinglong decoction, a san-huang gu-ben zhi-ke, a resveratrol, a P. cuspidatum, a lonicera extract, a houttuynia extract, a glycyrrhizae radix extract, a trichosanthis fructus extract, a parsnips extract, a mulberry leave extract, a mint extract, a perilla extract, a mumeplant extract, a poria extract, an atractylodes macrocephala extract, an agastache rugosa extract, a ganoderma extract, a curcuma extract, an astragalus extract, a phellinus linteus extract, an andrographis extract, an artemisia absinthium extract, a danshen extract, and a cinnamon extract.

In an embodiment of the present invention, the AGO particles are coated at least one nutritional product.

In an embodiment of the present invention, the AGO particles are coated at least one vaccine.

In an embodiment of the present invention, the AGO particles are made of an amphiphilic alginate.

In an embodiment of the present invention, an average diameter of each of AGO particles is form 100 nm to 500 nm.

In an embodiment of the present invention, each of the fog particles is an aerosol particle, and the aerosol particle comprises a concentration ranging from 0.001% to 5% by weight.

In an embodiment of the present invention, the aerosol particle is biocompatible and biodegradable.

To sum up, the substance delivery mask can encapsulate the water-soluble or water-insoluble medicines, or nutritional products in the fog particles generated from the atomizer. And the protection of the medicines and the nutritional products are not limited to the areas inside the body system, but those of the respiratory tract, including the nose, throat, and lungs.

### BRIEF DESCRIPTION OF THE DRAWINGS

FIG. 1 shows a schematic diagram of a substance delivery mask according to an embodiment of the present invention.

FIG. 2 shows a schematic diagram of the fog module according to the embodiment of the present invention.

FIG. 3 shows a schematic diagram of a substance delivery mask according to another embodiment of the present invention.

FIG. 4 shows a schematic diagram of the user inhaling the fog particles.

FIG. 5 shows slices of various organs after female rats inhaled the aerosol particles.

### DETAILED DESCRIPTION OF THE INVENTION

Reference will now be made in detail to embodiments illustrated in the accompanying drawings. Wherever possible, the same reference numbers are used in the drawings and the description to refer to the same or like parts. In the drawings, the shape and thickness may be exaggerated for clarity and convenience. This description will be directed in particular to elements forming part of, or cooperating more directly with, methods and apparatus in accordance with the present disclosure. It is to be understood that elements not specifically shown or described may take various forms well known to those skilled in the art. Many alternatives and modifications will be apparent to those skilled in the art, once informed by the present disclosure.

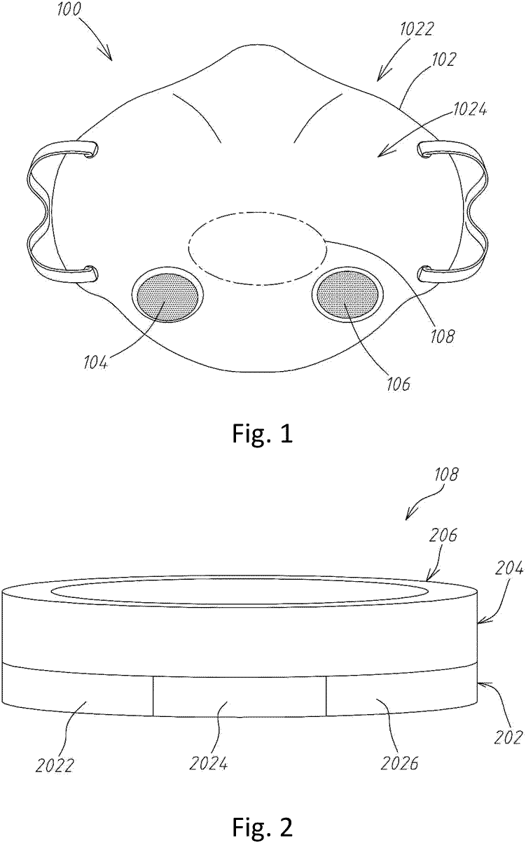

FIG. 1 is a schematic diagram of a substance delivery mask 100 according to an embodiment of the present invention. Please refer to FIG. 1. The substance delivery mask 100 is disposed a body 102, an exhaling valve 104, an inhaled valve 106, and a fog module 108. The body 102 has a first surface 1022 and a second surface 1024. The first surface 1022 and the second surface 1024 are located at the corresponding positions of the body 102. The exhaling valve 104 is disposed on the body 102. One end of the exhaling valve 104 is exposed to the first surface 1022, and another end of the exhaling valve 104 is exposed to the second surface 1024. The inhaled valve 106 is disposed on the body 102. One end of the inhaled valve 106 is exposed to the first surface 1022, and another end of the inhaled valve 106 exposed to the second surface 1024.

In addition, the substance delivery mask 100 can be a face mask, which covers the mouth and nose or only covers the mouth or nose. The substance delivery mask also can be a head mask, which covers the entire head. The body 102 of the substance delivery mask 100 is made of breathable material. The airproof material can be made of silicon, rubber, plastic, or a combination thereof. Moreover, the body 102 of the substance delivery mask 100 is also made of an airproof material. The airproof material can be made of a polypropylene nonwoven material, a melt-blown nonwoven material, a composite nonwoven material, or a combination thereof. The exhaling valve 104 and the inhaled valve 106 are one-way valves. Take the inhaled valve 106 as an example, the inhaled valve 106 is disposed a non-return valve. When the user inhales, the air pressure in the substance delivery mask 100 decreases, and the inhaled valve 106 opens. When the user exhales, the air pressure in the substance delivery mask 100 increases, and the inhaled valve 106 closes. The position of the non-return valve in the exhaling valve 104 is reversed. When the user exhales, the air pressure in the substance delivery mask 100 increases, and the exhaling valve 104 opens for the air into the substance delivery mask 100.

FIG. 2 is a schematic diagram of fog module 108 according to the embodiment of the present invention. Please refer to FIG. 2. The fog module 108 comprises a controlling module 202, a container 204, and an atomizer 206. The controlling module 202 is disposed on the first surface 1022. The container 204 is disposed above the controlling module 202. The atomizer 206 is disposed above the container 204. The controlling module 202 comprises a battery 2026, a programming module 2022, and a switch 2024.

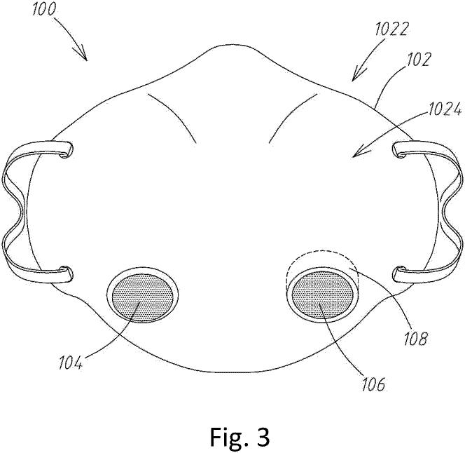

FIG. 3 is a schematic diagram of a substance delivery mask 100 according to another embodiment of the present invention. The end of the inhaled valve 106 is connected with the controlling module 202 of the fog module 108, and another end of the inhaled valve 106 connects with the body 102. The present invention does not limit the positions of the exhaling valve 104, the inhaled valve 106, and the fog module 108 on the body 102, and they can overlap or be placed at any position on the body.

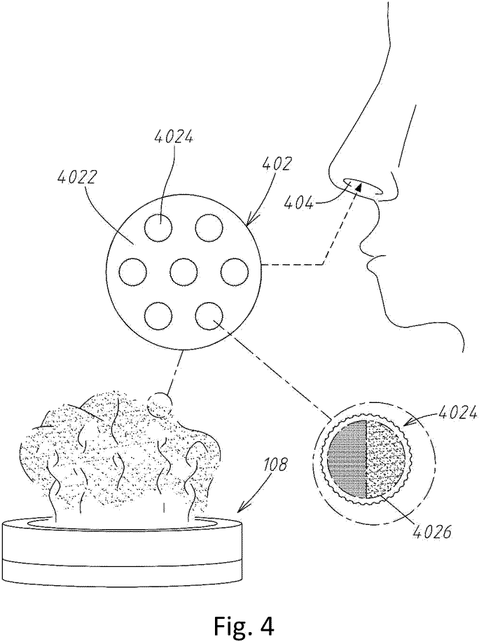

Please refer to FIG. 2 and FIG. 4. FIG. 4 is a schematic diagram of the user inhaling the fog particles. The container 204 stores a mixed liquid, and the mixed liquid comprises water 4022 and nanoparticles 4024. The battery 2026 connects with the programming module 2022, the switch 2024 connects between the battery 2026 and the programming module 2022. When the switch 2024 turns on, the battery 2026 powers the programming module 2022, and the programming module 2022 turns on and the atomizer 206 starts to shock. The mixed liquid in the container 204, which contacts with the atomizer 206 resonates by high-frequency vibration so that the mixed liquid is ejected from the holes of the atomizer 206 to form the fog particles 402. When the switch is turned off, it causes the power outage, and the fog particles 402 do not generate anymore. The fog particles 402 are the aerosol particles. The aerosol particles are biocompatible and biodegradable and can be taken by inhalation or oral. Each of the aerosol particles has a concentration in a range of 0.002% -7% by weight. Each of the fog particles 402 is wrapped in several of the nanoparticles 4024 in water 4022. In addition, the nanoparticles 4024 are the AGO particles 4024. The AGO particles 4024 are made of an amphiphilic alginate. The average diameter of each of the AGO particles 4024 is 100-500 nm.

The AGO particles 4024 are utilized an amphiphilic polysaccharide with controlled low-molecule weight termed as AGO amphiphile purchased from Nuecology Biomedical Inc. British Columbia, Canada. The AGO amphiphile functions as an amphiphilic alginate-based nanocarrier encapsulating vaccine as the active ingredient and adjuvants simultaneously. The AGO amphiphile shows a self-assembly behavior in aqueous solution to form spherical nanoparticles, excellent structural stability, colloidal stability for months, and biocompatibility in vitro and in vivo. The AGO amphiphile can form an AGO particle 4024 in an aqueous medium. The AGO amphiphile can be used as a biomedical material for multifunctional applications, such as a delivery system for an active agent. The AGO amphiphile includes but not limited to a drug, a biological agent or material, comprising a peptide, a protein, an antibody, a serum product, a vaccine, a plurality of cells or stem cells, or a combination of multiple active agents of various physicochemical properties, such as a mixture of water-soluble or lipid-soluble ingredients, or a combination of organic and inorganic active agents. The AGO particle 4024 is used to encapsulate lipophilic (water-insoluble) drugs or active ingredients. The AGO particle 4024 makes the content more water-soluble in the aqueous solution and enhances its bioavailability. The AGO particle 4024 stabilizes vulnerable drugs or active ingredients such as proteins or environmentally-sensitive active ingredients while nebulizing into aerosol microparticles for inhalation.

Each of the AGO particle 4024 is coated with at least one of the medicines, the nutrition product, the vaccines, or a combination of them. The medicines could be remdesivir, dexamethasone, favipiravir or avigan, hydroxychloroquine, carfilzomib, darunavi, pitavastatin, lamivudine, lopinavir, nelfinavir, ritonavir, darunavir, ledipasvir, telaprevir, rosuvastatin calcium, atovaquone, moexipril, azithromycin, curcumin, or artemisinin.

The medicines also could be Chinese medicines, such as a xiaoqinglong decoction, a san-huang gu-ben zhi-ke, a resveratrol, a P. cuspidatum, a lonicera extract, a houttuynia extract, a glycyrrhizae radix extract, a trichosanthis fructus extract, a parsnips extract, a mulberry leave extract, a mint extract, a perilla extract, a mumeplant extract, a poria extract, an atractylodes macrocephala extract, an agastache rugosa extract, a ganoderma extract, a curcuma extract, an astragalus extract, a phellinus linteus extract, an andrographis extract, an artemisia absinthium extract, a danshen extract, or a cinnamon extract.

The vaccines are coated in the AGO particles. The vaccines could be the composite-type nano-vaccine particle comprising an active ingredient, spike RBD protein (SEQ ID NO:1), two adjuvants as aluminum salt (Al(OH)₃) nanoparticle adsorbed with the spike RBD protein and synthetic oligonucleotides (CpG-ODN, SEQ ID NO:2), and an amphiphilic alginate-based nanocarrier (AGO amphiphile) encapsulating the active ingredient and the two adjuvants.

The following illustrations are the different experimental data of the present invention.

### Example 1: In-Vivo Evaluation on the Biosafety of the AGO Particles Carried Remdesivir Via Intranasal Aerosol Inhalation

1. Husbandry and test system: All animal experiments and care were approved by the Institutional Animal Care and Use Committee (IACUC) of the Agricultural Technology Research Institute(IACUC No. 109064) 7.1.1 Husbandry: Animals were housed in the AAALAC accredited facility of ATRI. Animals with the same gender and treatment were housed by using polycarbonate cages in the animal room

1.1 Temperature: 22±4° C.

1.2 Relative Humidity: 30~70%

1.3 Light Cycle: 12 hours light and 12 hours dark

1.4 Diets: Autoclaved laboratory 5053-PicoLab® Rodent Diet 20 -Lab Diet, Richmond, IN, USA

1.5 Water: Autoclaved RO water was supplied ad libitum viwater bottles attached to the cages

1.6 Identification: Animals were identified by ear notch and each cage was labeled with cage number, study number, IACUC number, gender, treatment, and animal ID number.

### 2 Test System

2.1 Strain and Source: Sprague Dawley strain / BioLASCO Taiwan Co. Ltd.

2.2 Age and body weight: At study initiation, rats were approximately 8 weeks old, with the body weights range of 177.43 g to 210.66 g prior to dosing

2.3 Sex/Number: Female/3

2.4 Acclimation: Animals were quarantined by GLP animal facility of ATRI. Animal Center and acclimated for 6 days in the testing room before dosing

3 Test Article Preparation: The “aerosol” was generated and processed as described above from a nebulizer, which can be equipped with a facemask to provide therapeutic function for infectious patients or booster health condition for elder. Test article was weighted and mixed with normal saline to be dissolved than diluted to 20 mg/mL before dosing.

### 4. Experimental Procedure

4.1 The animals were fasted for 16 to 18 hours before the intranasal administration, and three to four hours after the administration.

4.2 The administration was conducted with a nebulizer syringe at the volume of 0.5 mL/kg base on the body weight measured on the administration day.

4.3 Clinical Observation: The animals were observed continuously for 10 minutes after the administration, and observed 30 minutes and four hours after the administration on the administration day. The animals were observed once from 1 to 14 days after the administration.

4.4 Measurement of body weight: Body weights were measured 0 (before administration), 7 and 14 days after the administration with an electric balance.

4.5 Gross necropsy: The survived animals were subjected to a gross necropsy 14 days after the administration. The survived animals were euthanized by bleeding from the abdominal aorta under isoflurane anesthesia. External surface of the body, all orifices, subcutis, cranial, abdominal and pelvic cavities with their contents were observed for all animals.

4.6 Blood sampling and Treatment: Blood samples were obtained through abdominal aorta and collected in without the anticoagulants. Serum was harvested by centrifugation in a refrigerated centrifuge at 3,500 rpm for 15 minutes at 4° C. within 1 hour. Each serum samples were stored at -30° C.

4.7 Tissue Collecting and Organ Weight Measurements: The heart, lung, spleen, liver, kidney and adrenal gland were removed and weights were measured using an electric balance for all animals. The heart, lung, spleen, liver, kidney and adrenal gland were preserved in 10% neutralized buffered formalin.

### 5. Test Results

5.1 Mortality/Moribundity: No animal death was observed during the study period.

5.2 Clinical observations: No adverse clinical signs were observed in all animals during the study period.

5.3 Body Weight: No abnormalities were observed in all animals during the study period.

5.4 Gross necropsy findings: On the necropsy day, no abnormal findings were observed in all animals.

5.5 Organs weights: Individual animal organ weight data were monitored daily and no abnormal growth or change was observed.

### Example 2: Histopathological Analysis of the Vital Organs Prepared From Example 1

This study was entrusted by the Nuecology Biomedical Inc. Vancouver, BC, Canada. This study evaluated the pathological changes induced by the test article. The aerosol particles are coated AGO particles and water and AGO particles are coated medicine called AGO drugcarrying nanoparticles in the following description. AGO drugcarrying nanoparticles via intranasal administration in female rats. Three female Sprague Dawley rats, 8 weeks old, were dosed with the AGO containing aerosol (syringe nebulizer) at 0.5 mg/kg in double-distilled H₂O solution. All rats were sacrificed on day 14. The heart, kidneys, lungs, liver, and spleen were collected and were submitted for histopathological evaluation. Under histopathological evaluation, no significant lesions of the heart, kidneys, liver, lungs, or spleen were found in the aerosol treated females.

In conclusion, the pilot study of acute toxicity of at 0.5 mg/kg AGO-containing aerosol did not cause significant lesions of the heart, kidneys, liver, lungs, or spleen in female rats according to histopathological examination, as disclosed below:

### 1. Materials and Methods

Three female Sprague Dawley rats, 8 weeks old, were inhaled via intranasal administration with AGO containing aerosol at 0.5 mg/kg in double-distilled H₂O solution. All rats were sacrificed on day 14.

Please refer to Table 1. Table 1 is about pathological nomenclatures and criteria. The heart, kidneys, lungs, liver, and spleen were collected and were submitted for histopathological evaluation. Tissues were further processed, embedded in paraffin, cut at 3 Pm by microtone stained with Hematoxylin & Eosin (H&E) stain and evaluated under light microscope (BX-53, Olympus, Tokyo, Japan) for histopathological evaluation.

The severity of lesions was graded according to the methods described by Shackelford et al. (Toxicologic Pathology 30: 93-96, 2002). The degree of lesions was graded from one to five depending on severity: 1 = minimal (< 1%); 2: slight (1-25%); 3 = moderate (26-50%); 4 = moderately severe (51-75%); 5 = severe/high (76-100%).

### 2. Results

### 2.1 Histopathological Findings

### 2.1.1 Heart, Kidneys, Liver, Lungs, Or Spleen

Please refer to Table 2. Table 2 is about summary of pathological incidence of rats in the intranasal inhalation toxicity test of AGO-containing aerosol. No significant lesions of the heart, kidneys, liver, lungs, or spleen were found in the dosage of 0.5 mg/kg AGO™ treated group.

### 3. Conclusion

Three female Sprague Dawley rats, 8 weeks old, were intranasal inhalation dose at 0.5 mg/kg in double-distilled H₂O solution. All rats were sacrificed on day 14. The heart, kidneys, lungs, liver, lungs, and spleen were collected and were submitted for histopathological evaluation.

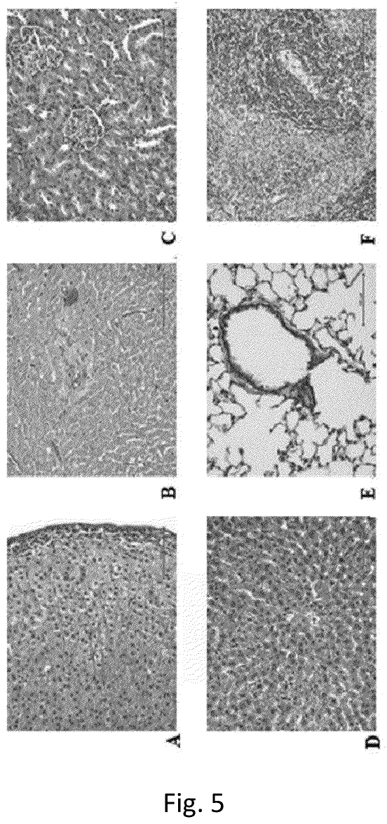

Please refer to Table 3 and FIG. 5. Table 3 is about pathology-individual micro findings of rats in the intranasal inhalation toxicity test of AGO-containing aerosol. FIG. 5 is slices of various organs after female rats inhaled the aerosol particles. From histopathological evaluation, no significant lesions of the heart, kidneys, liver, lungs, or spleen were found in the AGO concentration of 0.5 mg/kg treated female rats.

In conclusion, the pilot study of intranasal inhalation dose toxicity of aerosol with AGO particle at 0.5 mg/kg did not cause significant lesions of the heart, kidneys, liver, lungs, or spleen in female rats according to histopathological examination.

TABLE 1

Pathological nomenclatures and criteria
Observation fate:
Gross finding:
No abnormalities (NA)
Left (L); Right (R)
Bilateral (B)
Slight, +
Mild, ++
Moderate, +++
Severe, ++++

Histopathological nomenclatures:
No significant lesions (NSL)
Distribution: Focal, Multifocal, Local Extensive and Diffuse Degree¹
Slight, Moderate, and Severe
Duration: Acute, Subacute, and Chronic
Exudate: Serous, Fibrinous, and Purulent
Modification: Degeneration, Necrosis, ...
₁ Severity of lesions was graded according to the methods described by Shackelford et al. (2002) (Toxicologic Pathology 30: 93-96, 2002). Degree of lesions was graded from one to five depending on severity: 1 = minimal (< 1%); 2 = slight (1-25%); 3 = moderate (26-50%); 4 = moderate/severe (51-75%); 5 = severe/high (76-100%).

TABLE 2

Summary of pathological incidence of rats in the intranasal inhalation toxicity test of AGO™ -containing aerosol.

| Organ | Histopathological findings | Group |
|---|---|---|
| | | Aerosol with AGO™ concentration of 0.5 mg/kg |
| Heart | - | |
| Kidney | - | |
| Liver | - | |
| Lung | - | |
| Spleen | - | |

- No effect.
¹Degree of lesions was graded from one to five depending on severity: 1 = minimal (< 1%); 2 = slight (1-25%); 3 = moderate (26-50%); 4 = moderate/severe (51-75%); 5 = severe/high (76-100%).
²Incidence: Affected rats/ Total examined rats (n = 3)
³The final numerical score was calculated by dividing the sum of the number per grade of affected area by the total number of examined areas (n = 3)

TABLE 3

Pathology - individual micro findings of rats in the intransal inhalation toxicity test of AGO-containing aerosol

| Group/ organs | Histopathological findings | Animal code | | | | | | | | | | | |
|---|---|---|---|---|---|---|---|---|---|---|---|---|---|
| | | 0.5mg/kg | | | | | | | | | | | |
| | | 1 | 2 | 3 | / | / | / | / | / | / | / | / | / |
| Adrenal | | - | - | - | / | / | / | / | / | / | / | / | / |
| | | | | | / | / | / | / | / | / | / | / | / |
| | | | | | / | / | / | / | / | / | / | / | / |
| Heart | | - | - | - | / | / | / | / | / | / | / | / | / |
| | | | | | / | / | / | / | / | / | / | / | / |
| | | | | | / | / | / | / | / | / | / | / | / |
| Kidney | | - | - | - | / | / | / | / | / | / | / | / | / |
| | | | | | / | / | / | / | / | / | / | / | / |
| | | | | | / | / | / | / | / | / | / | / | / |
| Liver | | - | - | - | / | / | / | / | / | / | / | / | / |
| | | | | | / | / | / | / | / | / | / | / | / |
| | | | | | / | / | / | / | / | / | / | / | / |
| Lung | | - | - | - | / | / | / | / | / | / | / | / | / |
| | | | | | / | / | / | / | / | / | / | / | / |
| | | | | | / | / | / | / | / | / | / | / | / |
| Spleen | | - | - | - | / | / | / | / | / | / | / | / | / |
| | | | | | / | / | / | / | / | / | / | / | / |
| | | | | | / | / | / | / | / | / | / | / | / |

-: No significant lesions.
¹: Degree of lesions was graded from one to five depending on severity: 1 =minimal
(< 1%); 2 = slight (1–25%); 3= moderate (26–50%); 4 = moderate/severe (51–75%); 5 = severe/high (76–100%)

Please refer to FIG. 5. There is no significant change in (A) adrenal glands, (B) heart, (C) kidneys, (D) liver, (E) lung, and (F) spleen was found in the 0.5 mg/kg of AGO particle. It can be seen that the present invention can be directly inhaled through the mouth and nose, and is not toxic to animals. It shows that the present invention is safe, non-toxic, and easy to use.

### Example 3: In-Vivo Evaluation on the Biosafety of the Chinese Medicine-Carrying AGO Particles Via Intranasal Aerosol Inhalation

### 1. Chinese Medicinal Ingredients

A mixture of Chinese medicinal ingredients from a number of herbal extractions, includes a xiaoqinglong decoction, a san-huang gu-ben zhi-ke, a resveratrol, a P. cuspidatum, a lonicera extract, a houttuynia extract, a glycyrrhizae radix extract, a trichosanthis fructus extract, a parsnips extract, a mulberry leave extract, a mint extract, a perilla extract, a mumeplant extract, a poria extract, an atractylodes macrocephala extract, an agastache rugosa extract, a ganoderma extract, a curcuma extract, an astragalus extract, a phellinus linteus extract, an andrographis extract, an artemisia absinthium extract, a danshen extract, and a cinnamon extract.

### 2. Experimental Procedure

The AGO powder was dissolved and dispersed in the herbal solution with a concentration of 0.3 wt%, stirring for a time of 6 hours, to form a clear solution. The herbal extraction encapsulated in the AGO nanoparticle has a concentration ranging from 0.1% to 1%, and in a volume of 1 ml to 5 ml placed in the nebulizer for wearers’ use daily. The aqueous extraction of the mixtures can be used in a frequency of 2-8 times while nebulizing to form mist-like AGO-carrying aerosol particles within the space of the facemask for the wearer to inhale.

### 3. Test Results

The extraction mist evolved by the equipped nebulizer had shown to provide no any adverse effect to the animals and humans while inhaling and was considered to be biologically safe for practical uses. The herbal extractions employed in the example have long been shown to be protective of human lung diseases, and some of them may provide the cure for coronavirus infection in the clinical study.

The advantages of the present invention include (1) encapsulating lipophilic (water-insoluble) drug or active ingredient and make more water-soluble in the aqueous solution and enhance its bioavailability, (2) stabilizing vulnerable drug or active ingredients such as proteins or environmentally-sensitive active ingredient while nebulizing into aerosol microparticles for inhalation, (3) to provide sustained release of the drug or active ingredient after the aerosol particles being inhaled into the respiratory tracts of the wearers, i.e., reduce the inhaled frequency for the wearer, and (4) to prolong the lifetime of therapeutic performance and reduce the dosing frequency, for instance, 2-3 times per day in case needed.

The embodiments described above are only to exemplify the present invention and should not be limited to the scope of the present invention. Therefore, any equivalent modification or variation according to the shapes, structures, features, or spirit disclosed by the present invention is to be also included within the scope of the present invention.

What is claimed is:

1. A substance delivery mask, comprising:
a body having a first surface and a second surface opposite to each other;
an exhaling valve disposed on the body, wherein a first end of the exhaling valve is exposed to the first surface, and a second end of the exhaling valve is exposed to the second surface;
an inhaled valve disposed on the body, wherein a first end of the inhaled valve is exposed to the first surface, and a second end of the inhaled valve is exposed to the second surface; and
a fog module comprising:
a controlling module disposed on the first surface;
a container disposed on the controlling module and storing a mixed liquid, wherein the mixed liquid comprises water and a plurality of nanoparticles; and
an atomizer disposed on the container and configured to atomize the water of the mixed liquid to form a plurality of fog particles, wherein the fog particles are respectively wrapped in the nanoparticles.

2. The substance delivery mask of claim 1, wherein the body is made of airproof material.

3. The substance delivery mask of claim 2, wherein the airproof material comprises silicon, rubber, plastic, or a combination thereof.

4. The substance delivery mask of claim 1, wherein the body is made of breathable material.

5. The substance delivery mask of claim 4, wherein the breathable
material comprises at least one of polypropylene nonwoven material,
melt-blown nonwoven material, and composite nonwoven material.

6. The substance delivery mask of claim 1, wherein the controlling module comprises a battery, a programming module, and a switch, wherein the battery is connected with the programming module, and the switch is connected between the battery and the programming module.

7. The substance delivery mask of claim 1, wherein the first end of the inhaled valve is connected with the controlling module and the second end of the inhaled valve is connected with the body.

8. The substance delivery mask of claim 1, wherein each of the nanoparticles is an AGO particle.

9. The substance delivery mask of claim 8, wherein each of the AGO particles is coated with at least one medicine.

10. The substance delivery mask of claim 9, wherein the at least one medicine comprises at least one of a remdesivir, a dexamethasone, a favipiravir, an avigan, a hydroxychloroquine, a carfilzomib, a darunavi, a pitavastatin, a lamivudine, a lopinavir, a nelfinavir, a ritonavir, a darunavir, a ledipasvir, a telaprevir, a rosuvastatin calcium, an atovaquone, a moexipril, an azithromycin, a curcumin, and an artemisinin.

11. The substance delivery mask of claim 9, wherein the at least one medicine comprises at least one of a xiaoqinglong decoction, a San-huang gu-ben zhi-ke, a resveratrol, a P. cuspidatum, a lonicera extract, a houttuynia extract, a glycyrrhizae radix extract, a trichosanthis fructus extract, a parsnips extract, a mulberry leave extract, a mint extract, a perilla extract, a mumeplant extract, a poria extract, an atractylodes macrocephala extract, an agastache rugosa extract, a ganoderma extract, a curcuma extract, an astragalus extract, a phellinus linteus extract, an andrographis extract, an artemisia absinthium extract, a danshen extract, and a cinnamon extract.

12. The substance delivery mask of claim 8, wherein each of the AGO particles is coated with at least one nutritional product.

13. The substance delivery mask of claim 8, wherein each of the AGO particles is coated with at least one vaccine.

14. The substance delivery mask of claim 8, wherein each of the AGO particles is made of amphiphilic alginate.

15. The substance delivery mask of claim 8, wherein an average diameter of each of AGO particles is from 100 nm to 500 nm.

16. The substance delivery mask of claim 15, wherein each of the fog particles is an aerosol particle and the aerosol particle comprises a concentration ranging from 0.001% to 5% by weight.

17. The substance delivery mask of claim 16, wherein the aerosol particle is biocompatible and biodegradable.
